(19) United States
(12) Patent Application Publication
(10) Pub. No.: US 2012/0028891 A1
(43) Pub. Date: Feb. 2, 2012

(54) METHODS OF PROMOTING OR MAINTAINING AN ANABOLIC STATE IN A FEMALE ANIMAL

(75) Inventors: Inke Paetau-Robinson, Auburn, KS (US); Jeffrey Brockman, Lawrence, KS (US)

(73) Assignee: Hill's Pet Nutrition, Inc., Topeka, KS (US)

(21) Appl. No.: 13/193,840

(22) Filed: Jul. 29, 2011

Related U.S. Application Data
(60) Provisional application No. 61/368,750, filed on Jul. 29, 2010.

(51) Int. Cl. A61K 38/00

(52) U.S. Cl. 514/4.8; 514/1.1; 514/16.5; 514/16.7

(57) ABSTRACT

The present invention is related to methods of promoting or maintaining an anabolic state in a female animal. The methods comprise administering a composition to the female animal, with the composition comprising between about 15% to about 25% protein (w/w) and about 5% to about 15% fat (w/w). The protein source in the composition comprises chicken eggs and further comprises a second protein source selected from the group consisting of soybean meal and lamb meat

### CROSS-REFERENCE TO RELATED APPLICATIONS

This application claims priority to U.S. Provisional Patent Application No. 61/368,750, which was filed Jul. 29, 2010, which is incorporated herein by reference in its entirety.

### BACKGROUND OF THE INVENTION

In the past few years, pet nutrition has come to the forefront of domestic pet care. In particular, many different food and dietary compositions have been created to address special needs and conditions in domestic companion animals such as cats and dogs. For example, different companies have produced pet foods designed to reduce caloric intake, or reduce gastrointestinal tract irritation or maintain a healthy spectrum of gut microflora. Other compositions include compositions designed to stabilize blood glucose and even reduce inflammatory responses in older arthritic animals.

To date, however, no such domestic pet food compositions address different metabolic needs of the animal that may be gender-based. The inventors have surprisingly found that male and female domestic companion animals of roughly the same age exhibit different metabolic profiles after ingesting the same diet. The inventors, in response, have discovered several novel pet food compositions that address these gender-specific metabolic profiles in domestic companion animals.

### SUMMARY OF THE INVENTION

The present invention encompasses methods of maintaining an anabolic state in a female companion animal. The methods comprise administering a food composition, including a nutritionally complete dietary composition, to the female animal, with the composition comprising between about 15% to about 25% protein (w/w) and about 5% to about 15% fat (w/w). The protein source in the composition comprises chicken eggs and further comprises a second protein source selected from the group consisting of soybean meal and lamb meat.

The present invention also encompasses methods of promoting an anabolic state in a female companion animal. The methods comprise administering a composition to the female animal, with the composition comprising between about 15% to about 25% protein (w/w) and about 5% to about 15% fat (w/w). The protein source in the composition comprises chicken eggs and further comprises a second protein source selected from the group consisting of soybean meal and lamb meat.

The present invention additionally encompasses methods for promoting and maintaining an anabolic state in a female companion animal by administering a food composition, including a nutritionally complete dietary composition, to such female companion animal, which food composition comprises a protein source wherein the protein source in the composition comprises chicken eggs and further comprises a second protein source selected from the group consisting of soybean meal and lamb meat in an amount of about 15% to about 25% protein (w/w) and a fat source in an amount of about 5% to about 15% fat (w/w).

### DETAILED DESCRIPTION

As used throughout, ranges are used as a shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

The present invention encompasses methods of promoting or maintaining an anabolic state in a female animal.

In one embodiment the female animal is a companion animal, for example, a canine or a feline.

In another embodiment, the method comprises administering a food composition comprising a protein source present in an amount of about 15% by weight to about 25% by weight of the composition and a fat source present in an amount of about 5% to about 15% by weight of the composition.

In one embodiment, the protein source of the food composition comprises chicken eggs and soybean meal.

In another embodiment, the protein source of the food composition comprises chicken eggs and lamb meat.

In another embodiment, the protein source of the food composition comprises chicken eggs, soybean meal and lamb meat.

In yet another embodiment, the protein source of the food composition comprises corn and poultry meal and one or more additional protein sources wherein the protein source in the composition comprises chicken eggs and further comprises a second protein source selected from the group consisting of soybean meal and lamb meat.

In yet another embodiment, the food composition further includes one or more additional sources of proteins, fats, carbohydrates, fibers, and combinations thereof.

In still another embodiment, the food composition is a nutritionally complete dietary food composition, a supplement, an animal treat, or a toy.

In still another embodiment, the food composition is in the form of a moist food, dry food, supplement or treat.

### DEFINITIONS

It is contemplated that the invention described herein is not limited to the particular methodology, protocols, ingredients and reagents described, as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All publications mentioned herein are incorporated by reference for the purpose of describing and disclosing the materials and methodologies that are reported in the publication, which might be used in connection with the invention.

As used herein and in the appended claims, the singular forms “a”, “an”, and “the” include plural reference unless the context clearly dictates otherwise.

As used herein, the term “animal” is a non-human animal, and the term includes domesticated animals, such as house pets, laboratory animals, such as primates, and livestock. In one embodiment, the animal is a non-primate mammal. In another embodiment, the animal is a non-human primate. In a more specific embodiment, the animal is a domesticated companion animal or “house” pet, such as a female feline or female canine.

As used herein, the term “anabolic state” is characterized by at least one of tissue building, mineralization of bone, increase or maintenance of muscle mass and weight gain or weight maintenance. An overall catabolic state is characterized by at least one of tissue breakdown, a lack or decrease in bone mineralization, a decrease in muscle mass and weight loss. The invention is directed to methods of promoting or maintaining an overall anabolic state in a female animal, which means that the anabolic processes are functioning to a greater extent than the catabolic processes.

Determining if the anabolic processes are functioning to a greater extent than the catabolic processes can be assessed by reviewing the metabolic profile of the animal. Assessing the metabolic profile of the animal can be accomplished by analyzing blood, serum, plasma, urine, feces, tissue samples and even expired air from the animal. For example, whole blood can be withdrawn from the animal and analyzed for levels of amino acids and/or metabolites thereof, types of amino acids and/or metabolites thereof, levels of carbohydrates and/or metabolites thereof, types of carbohydrates and/or metabolites thereof, levels of lipids and/or metabolites thereof, types of lipids and/or metabolites, levels of fatty acids and/or metabolites thereof, types of fatty acids and/or metabolites, levels of organic acids and/or metabolites thereof, types of organic acids and/or metabolites, levels of vitamins and/or metabolites thereof, types of vitamins and/or metabolites thereof, levels of cofactors and/or metabolites thereof, types of cofactors and/or metabolites thereof. Thus, assessing the metabolic profile of the animal can be a qualitative assessment or a quantitative assessment, or a combination thereof.

The metabolic profile of an animal can be assessed by determining levels of one or a battery of serum markers that include specific compounds and/or their metabolites. Methods of determining serum marker levels include but are not limited to NMR, spectrophotometry, including mass spectrometry, gas and liquid chromatography, mass radiography, electrophoresis, capillary electrophoresis, high performance liquid chromatography (HPLC), thin layer chromatography (TLC), hyperdiffusion chromatography, various immunological methods, immunodiffusion, immunoelectrophoresis, radioimmunoassays (RIAs), enzyme-linked immunosorbent assays (ELISAs), immuno-fluorescent assays, Northern analysis, Western Analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

The term “antioxidant” means a substance that is capable of reacting with free radicals and neutralizing them. Illustrative examples of such substances include beta-carotene, selenium, coenzyme Q10 (ubiquinone), lutein, tocotrienols, soy isoflavones, S-adenosylmethionine, glutathione, taurine, N-acetylcysteine, vitamin E, vitamin C, lipoic acid and L-carnitine. Examples of foods containing useful levels of one or more antioxidants include but are not limited to, ginkgo biloba, green tea, broccoli, citrus pulp, grape pomace, tomato pomace, carrot spinach, and a wide variety of fruit meals and vegetable meals. It will be understood by one of skill in the art that while units of antioxidants may be provided herein as “ppm”, appropriate amounts of antioxidants may also be provided as “IU/kg” where appropriate and customary for a given antioxidant such as, e.g., Vitamin E.

The term “carbohydrate” as used herein includes polysaccharides (e.g., starches and dextrins) and sugars (e.g., sucrose, lactose, maltose, glucose, and fructose) that are metabolized for energy when hydrolyzed. Examples of carbohydrates suitable for inclusion in the compositions disclosed herein include, but are not limited to, corn, grain sorghum, wheat, barley, and rice.

The term “cat” includes those cats which are companion animals known as domestic cats or house cats, or Felis domesticus. The term cat is synonymous with the term feline.

The term “companion animal” used in the present invention includes any non-human animal suitable for being kept as a pet by humans including a dog, a cat, and a rodent. All aspects of the present invention are preferably for the treatment of cats and/or dogs.

The term “dog” includes those dogs which are companion animals such as Canis familiaris, working dogs and the like. The term dog is synonymous with the term canine.

As used herein, “an amount effective”, “an effective amount”, and like terms refer to that amount of a compound, material or composition as described herein that may be effective to achieve a particular biological result. The response to effective amount may be a cellular, organ or tissue-specific response, or system response, such as the reduction or maintenance in the levels of a risk factor or biomarker associated with an abnormal condition. Such effective activity may be achieved, for example, by administration of compositions of the present invention to an animal. An effective amount may be based on several factors, including an animal's ideal weight, the metabolizable energy of the composition, and frequency of feeding the animal compositions of the present invention, e.g., once, twice, or three times daily, and other compositions fed to the animal.

A “food” can, in some embodiments of the invention, be a nutritionally complete diet for the intended recipient animal (e.g., domestic cat or domestic dog).

As used herein, an “ingredient” refers to any component of a composition.

As used herein, “maintaining an anabolic state” in an animal is used to indicate that the metabolic status of the animal, prior to administration of the compositions of the present invention, is an overall anabolic metabolism and the compositions keep the animal in an overall anabolic state. Thus, a healthy female animal could, in one embodiment of the present invention, consume the compositions of the present invention to maintain her overall health.

The term “nutrient” refers to a substance that provides nourishment. In some cases an ingredient may comprise more than one “nutrient,” for example, a composition may comprise fish oil as an ingredient, the oil itself comprising important nutrients such as eicosapentaenoic acid and docosahexaenoic acid. The distinction in these terms is familiar to one of skill in the art.

As contemplated herein, the compositions of the present invention are meant to encompass nutritionally complete and balanced animal food compositions. A “nutritionally complete diet” is a diet that includes sufficient nutrients for maintenance of normal health of a healthy animal on the diet. Nutritionally complete and balanced pet food compositions are familiar to one of skill in the art. For example substances such as nutrients and ingredients suitable for nutritionally complete and balanced animal feed compositions, and recommended amounts thereof, may be found for example, in the Official Publication of the Association of American Feed Control Officials, Inc. (AAFCO), Atlanta, Ga., (2005).

The term “oxidative stress” refers to an imbalance between the production of reactive oxygen species and a biological system's ability to readily detoxify the reactive intermediates or easily repair the resulting damage. Reactive oxygen species include super oxide, ozone, peroxide and a hydroxyl radical. Oxidative stress causes cellular damage through mechanisms such as damage of DNA, oxidation of polydesaturated fatty acids in lipids, oxidation of amino acids in proteins, and inactivation of specific enzymes by oxidation of co-factors.

As used herein, “promoting an anabolic state” in an animal is used to indicate that the metabolic status of the animal is less catabolic than if the animal had not received the compositions of the present invention. Thus, “promoting an anabolic state” can mean changing the metabolic state in an animal from an overall catabolic state to an overall anabolic state. “Promoting an anabolic state” can also mean making an animal “less catabolic.” For example, elevated levels of urea and decreased levels of ascorbic acid in the serum can be markers for a catabolic metabolism, and “promoting an anabolic state,” as used herein, would mean that the serum levels of urea are reduced and ascorbic acid levels are increased in animals. In another example, reduction in the amount of triacylglycerols in the female animal in response to the administrations of the compositions of the invention is indicative of promoting an anabolic state in said female animal. Thus, one aspect of the present invention is directed to altering the metabolic profile of a female animal, comprising administering the compositions of the present invention to the animal. Promoting an anabolic state in an animal could also be used to indicate that an animal that was previously undernourished would begin to gain weight in response to consumption of the compositions of the present invention.

The terms “sample” and “specimen” mean any animal tissue or fluid containing, e.g., polynucleotides, polypeptides, antibodies, metabolites, and the like, including cells and other tissue containing DNA and RNA. Examples include: blood, cartilage, connective, epithelial, lymphoid, muscle, nervous, sputum, and the like. A sample may be solid or liquid and may be DNA, RNA, cDNA, bodily fluids such as blood or urine, cells, cell preparations or soluble fractions or media aliquots thereof, chromosomes, organelles, and the like.

As used herein, “soluble fiber” refers to dietary fiber that attracts water during digestion and slows the rate of nutrient absorption and is typically found in, e.g., oat bran, seeds, beans, and certain fruits and vegetables such as beet pulp, guar gum, chicory root, psyllium, pectin, blueberry, cranberry, squash, apples, oats, beans, citrus, barley and peas. As used herein, the term encompasses any source of soluble fiber suitable for the compositions disclosed herein as would be evident to one of skill in the art.

The term “substance” means an element, compound, molecule, or a mixture thereof or any other material that could potentially be useful for diagnosing or prognosing the anabolic state of an animal, including any drug, chemical entity, or biologic entity.

As used herein, the term “supplement(s)” include, but are not limited to, a feed used with another feed to improve nutritive balance or performance of the total diet for an animal. Supplements include, but are not limited to, compositions that are fed undiluted as a supplement to other feeds, offered free choice with other parts of an animal's ration that are separately available, or diluted and mixed with an animal's regular feed to produce a complete feed. The AAFCO guidelines, for example, contain a discussion relating to supplements in the Official Publication of the Association of American Feed Control Officials, Inc. (AAFCO), Atlanta, Ga. (2005). Supplements may be in various forms including, for example, powders, liquids, syrups, pills, encapsulated compositions and the like.

For example, a nutritionally complete and balanced dog food composition of the present invention may comprise: about 0 to about 90%, preferably about 5% to 60%, by weight of carbohydrates; about 5% to about 70%, preferably about 15% to about 25%, by weight of protein; about 2% to about 50%, preferably about 5% to about 15%, by weight of fat; about 0.1% to about 40%, preferably about 1% to about 11%, by weight of total dietary fiber; about 0 to about 15%, preferably about 2% to about 8%, by weight of vitamins and minerals, antioxidants, and other nutrients which support the nutritional needs of the animal.

For example, a nutritionally complete and balanced cat food composition of the present invention may comprise: about 0 to about 90%, preferably about 5% to 60%, by weight of carbohydrates; about 5% to about 70%, preferably about 15% to about 25% by weight of protein; about 2% to about 50%, preferably about 5% to about 15%, by weight of fat; about 0.1% to about 20%, preferably about 1% to about 11%, by weight of total dietary fiber; about 0 to about 15%, preferably about 2% to about 8%, by weight of vitamins and minerals, antioxidants, and other nutrients which support the nutritional needs of the animal.

All percentages expressed herein are by weight of the composition on dry matter basis unless specifically stated otherwise.

### Metabolomic Markers of the Invention

Metabolite profiling of the animals fed the test food compositions of the present invention can be performed by various methods previously stated herein and as known by a person of ordinary skill in the art. Target metabolites may be measured by such methods and can include the following classes and types of substances.

Specific non-limiting examples of amino acids and/or their metabolites include but are not limited to alanine, arginine, ornithine, asparagine, aspartate, citrulline, cysteine, glutamate, glutamine, glycine, isoleucine, leucine, lysine, methionine, N,N-dimethylarginine, O-phospho-L-tyrosine, phenylalanine, proline, serine, taurine, threonine, trans-4-hydroxyproline, tryptophan, tyrosine, valine, kynurenic acid, 3-hydroxyindole, 3-indoxylsulfuric acid, 4-hydroxyphenylpyruvic acid and urea.

Specific non-limiting examples of carbohydrates and/or their metabolites include but are not limited to 1,5-anhydrosorbitol, glucose-6-phosphate, fructose-6-phosphate, galactose, glucose, glucose-6-phosphate, glucose polar fraction, mannose and myo-inositol.

Specific non-limiting examples of organic acids and/or their metabolites include but are not limited to alpha-ketoglutaric acid, alpha-ketoisocaproic acid, citrate, lactate, malate, pyruvate, succinate and 3-hydroxybutyric acid.

Specific non-limiting examples of vitamins and cofactors and/or their metabolites include but are not limited to, alpha-tocopherol, ascorbic acid, coenzyme Q10, flavine-adenine dinucleotide, pantothenic acid and threonic acid.

Specific non-limiting examples of lipids and/or fatty acids and/or their metabolites include but are not limited to, glycerol, arachadonic acid, cholesterol, diacyl-glycerol, dihydrocholesterin, docosaheaxnoic acid, dodecanol, eicosanoic acid, eicosapentaenoic acid, elaidic acid, gamma-linoleic acid, glycerol lipid fraction, glycerophosphate, henicosanoic acid, heptadecanoic acid, lignoceric acid, linoleic acid, linolenic acid, methylgalactopyranoside, myo-inositol-2-monophosphate, myristic acid, nervonic acid, oleic acid, palmitic acid, stearic acid, palmitoleic acid, pentadecanol, phosphate (lipids), phospholipids (cholines), triacylglycerol, sphingosine, sphingosin derivatives and tricosanoic acid.

Specific non-limiting examples of other compounds and/or their metabolites include but are not limited to, creatine, creatinine, glycerol-3-phosphate, phosphate (inorganic and organic), progesterone, thyroxine, allantoin, beta-sitosterol, and campesterol.

Other methods of determining if an animal is in an overall anabolic state, versus a catabolic state, would be to measure and monitor the weight of the animal. A veterinarian would be well-suited to determine a healthy weight in an animal based upon such factors as, but not limited to, the breed, sex, age, nutritional state and body type of the animal. For example, a healthy weight in an animal may be a dog with less than 10% body fat or within a certain weight range for the dog's age, skeletal frame, musculature and breed. The invention is not limited by the means of assessing the metabolic state of the animal.

### Food Compositions

The invention is based upon the novel discovery that male and female companion animals of roughly the same age exhibit different metabolic profiles after ingesting the same diet. The inventors have discovered that the food compositions of the invention, including nutritionally complete dietary compositions formulated specifically for female companion animals, including dogs and cats, may promote and/or maintain an anabolic state in such female animal.

Formulating gender-based pet food compositions to promote and/or maintain an anabolic state in such animal was not heretofore appreciated by the skilled worker. The inventors herein disclose methods of promoting and/or maintaining an anabolic state in a female animal by administering a nutritionally complete dietary composition to such female animal.

The invention generally encompasses pet food compositions for a female companion animal comprising one or more source of protein selected wherein the protein source in the composition comprises chicken eggs and further comprises a second protein source selected from the group consisting of soybean meal and lamb meat in an amount of about 15% to about 25% by weight, a source of fat in an amount of about 5% to about 15% by weight, and optionally, an amount of carbohydrate, and/or an amount of dietary fiber, and/or vitamins and minerals, antioxidants, and/or other nutrients which support the nutritional needs of the female animal.

The methods of the invention comprise administering a composition to a female animal, the composition comprising between about 15% to about 25% protein (w/w). In specific examples, the concentration of protein is from about 17% to about 20% (w/w) of the total composition. In another specific embodiment, the concentration of protein is from about 18% to about 19% (w/w). In an even more specific embodiment, the concentration of protein is about 18.5% (w/w) of the total weight.

The methods of the invention also comprise administering a nutritionally complete dietary composition to a female animal, the composition comprising between about 15% to about 25% protein (w/w). In specific examples, the concentration of protein is from about 17% to about 20% (w/w) of the total composition. In another specific embodiment, the concentration of protein is from about 18% to about 19% (w/w). In an even more specific embodiment, the concentration of protein is about 18.5% (w/w) of the total weight.

For example, a nutritionally complete and balanced dog food composition of the present invention may comprise: about 0 to about 90%, preferably about 5% to 60%, by weight of carbohydrates; about 5% to about 70%, preferably about 15% to about 25%, by weight of protein; about 2% to about 50%, preferably about 5% to about 15%, by weight of fat; about 0.1% to about 40%, preferably about 1% to about 11%, by weight of total dietary fiber; about 0 to about 15%, preferably about 2% to about 8%, by weight of vitamins and minerals, antioxidants, and other nutrients which support the nutritional needs of the animal.

For example, a nutritionally complete and balanced cat food composition of the present invention may further comprise: about 0 to about 90%, preferably about 5% to 60%, by weight of carbohydrates; about 5% to about 70%, preferably about 15% to about 25% by weight of protein; about 2% to about 50%, preferably about 5% to about 15%, by weight of fat; about 0.1% to about 20%, preferably about 1% to about 11%, by weight of total dietary fiber; about 0 to about 15%, preferably about 2% to about 8%, by weight of vitamins and minerals, antioxidants, and other nutrients which support the nutritional needs of the animal.

In one embodiment, the female canine compositions of this invention include ingredients in an amount effective to promote an anabolic state in such female canine. Such compositions generally comprise:

(a) about 15 wt. % to about 25 wt. % of crude protein on a dry matter basis,

(b) about 5 wt. % to about 15 wt. % of fat on a dry matter basis, and

(c) about 1 wt. % to about 5 wt. % of crude fiber on a dry matter basis.

In another embodiment, the invention encompasses female feline compositions that include ingredients in an amount effective to promote an anabolic state in such female feline and which generally comprise:

(a) about 18.5% wt. % of crude protein on a dry matter basis,

(b) about 8 wt. % to about 10 wt. % of fat on a dry matter basis, and

(c) about 1 wt. % to about 5 wt. % of crude fiber on a dry matter basis.

In certain embodiments, the compositions of the invention can include an omega-3 polyunsaturated fatty acid content of at least about 0.02% (or about 0.05% to about 10%, or about 0.1% to about 6%) by weight on a dry matter basis. In some embodiments, the omega-3 polyunsaturated fatty acid is DHA. In other embodiments, the omega-3 polyunsaturated fatty acid is EPA. In still other embodiments, the omega-3 polyunsaturated fatty acid comprises a mixture of DHA and EPA.

The compositions of the present invention comprise at least one protein source wherein the protein source in the composition comprises chicken eggs and further comprises a second protein source selected from the group consisting of soybean meal and lamb meat.

In one embodiment, the protein source is chicken eggs. In another embodiment, the protein source is soybean meal and chicken eggs. In another embodiment, the protein source is soybean meal and chicken eggs. In yet another embodiment, the protein source is soybean meal, lamb meat and chicken eggs. In still another embodiment, the protein source is lamb meat. In another embodiment, the protein source is lamb meat and chicken eggs. In another embodiment, the protein source is chicken eggs. If more than one of the listed protein sources is used, the amounts of each individual protein source can be equal or unequal. For example, if soybean meal and lamb meat are used as the protein source, each one may be used in a 1:1 ratio (w/w) or the ratio of one ingredient to another may be higher or lower. If all three sources are used, then, in a likewise fashion, the amounts used may be in a 1:1:1 ration (w/w/w). Alternatively, the ratios of each source may be higher or lower.

The compositions may also comprise an additional protein source. Any suitable protein source may be used as the additional protein source. Suitable protein sources may be selected from any suitable animal or vegetable protein source, for example muscular or skeletal meat, meat and bone meal, poultry meal, fish meal, milk proteins, corn, corn meal, corn gluten, wheat gluten, soy flour, soy protein concentrates, soy protein isolates, egg proteins, whey, casein, gluten, and the like. Animal sources include, for example, meat, meat by-products, seafood, dairy, eggs, etc. Meats include, for example, the flesh of poultry, fish, and mammals (e.g., cattle, pigs, sheep, goats, and the like). Meat by-products include, for example, lungs, kidneys, brain, livers, and stomachs and intestines (freed of all or essentially all their contents). The protein can be intact, almost completely hydrolyzed, or partially hydrolyzed. The amount of “crude protein” in a composition disclosed herein may be determined based on the amount of nitrogen in the composition according to methods familiar to one of skill in the art.

The compositions of the present invention may also include amino acids in amounts required to avoid deficiency and maintain health. These amounts and methods of measurement are known by those skilled in the art. For example, AAFCO provides recommended amounts of such ingredients for dogs and cats. Amino acids in the present compositions may be supplied by any number of sources, including crude protein, or addition of free amino acids to the composition.

In certain embodiments, the fat and carbohydrate food ingredient is obtained from a variety of sources such as animal fat, fish oil, vegetable oil, meat, meat by-products, grains, other animal or plant sources, and mixtures thereof. Grains include wheat, corn, barley, and rice.

The compositions of the present invention also comprise about 5% to about 15% fat. In one embodiment, the fat may comprise long chain fatty acids. Examples of suitable long chain fatty acids include alpha-linoleic acid, gamma linolenic acid, linoleic acid, eicosapentaenoic acid (EPA), and docosahexaenoic acid (DHA), including suitable salts thereof. Fish oils are a suitable source of eicosapentaenoic acids and docosahexaenoic acid. Borage oil, blackcurrant seed oil and evening primrose oil are suitable sources of gamma linolenic acid. Safflower oils, sunflower oils, corn oils and soy bean oils are suitable sources of linoleic acid. Non-limiting examples of omega-3-fatty acid sources include sources such OMEGAPURE, commercially available from Omega Protein, Inc., Houston, Tex. All forms of the fatty acid are also contemplated herein. For example, DHA is often provided as a triglyceride. As such, wherein a specific fatty acid is mentioned (e.g., “DHA”), such fatty acid includes the free form of the fatty acid as well as other forms such as the naturally occurring triglyceride or other form. The terms, DHA, EPA, or other specific terms are utilized for convenience as will be commonly understood in the art to include all forms of such termed material. In one embodiment, the compositions comprise both EPA and DHA.

Other examples of suitable fatty acids may include oleic acid, stearic acid, palmitic acid, and lauric acids, including suitable salts thereof. Even further examples of suitable fatty acids include esters or other derivatives thereof, such as cetyl palmitate, acetic, lactic, or citric mono- and di-glyceride fatty acids, isopropyl palmitate, isopropylmyristate, and mono-, di-, and triglycerides (some of which may also be characterized as fats).

The fat component may also comprise a mixture of omega-3-fatty acids and omega-6-fatty acids, often through utilization of various materials containing these components. In addition, the compositions may optionally be formulated with a specific weight ratio of n-6 (omega-6) fatty acids to n-3 (omega-3) fatty acids (“n-6:n-3 ratio”). Of course, one of skill in the art will recognize various types of omega-3 and omega-6 fatty acids. For example, α-linolenic acid, stearidonic acid (STD) eicosatrienoic acid (ETE) eicosatetraenoic acid (ETA), EPA, docosapentaenoic acid (all-cis-7,10,13,16,19-docosapentaenoic acid) (a/k/a clupanodonic acid), DHA, tetracosapentaenoic acid and tetracosahexaenoic acid (Nisinic acid) are all known omega-3 fatty acids. Examples of omega-6 fatty acids include, but are not limited to, linoleic acid, γ-linolenic acid, eicosadienoic acid, dihomo-gamma-linolenic acid, arachidonic acid, docosadienoic acid, adrenic acid, docosapentaenoic acid (all-cis-4,7,10,13,16-docosapentaenoic acid) (a/k/a osbond acid) and calendic acid.

The composition may also optionally comprise additional fats, or lipids, beyond including EPA and DHA. Of course, the fat content as well as other components of the composition will provide an energy source. In select embodiments, the fat content will be the major source for energy in the compositions. Suitable lipid sources include meats, animal fats and vegetable fats. The lipid component may be any component comprising a source of fat, defined herein to be inclusive of, for example, wax, fat, fatty acid, and lipid. Specific examples of wax, fat, fatty acid, or lipid may often be interchangeable in accordance with nomenclature common in the art; for example, a lipid may often also be characterized as a fat. The invention is not limited by any particular designation of nomenclature, and classifications of a particular material as a wax, fat, fatty acid, lipid, or the like. In one embodiment, the amount of total fat in the compositions is from about 5% to about 15% (w/w). In additional embodiments, the amount of fat in the compositions is from about 7% to about 12% or from about 9% to about 12%.

Alternatively or additionally, the lipid component may comprise an animal-derived fat component. As will be commonly known in the art, the animal-derived fat component comprises a fat derived from an animal. Non-limiting examples include beef, poultry, pork, and lamb (e.g., lards and tallows). Dairy fats may also be examples, including milkfat, fractionated milkfat, and butterfat.

In certain embodiments, the food compositions may contain nutritional balancing agents. The nutritional balancing agents are obtained from a variety of sources known to skilled artisans, for example, vitamin and mineral supplements and food ingredients. Vitamins and minerals can be included in amounts required to avoid deficiency and maintain health. These amounts are readily available in the art. The Association of American Feed Control Officials (AAFCO) provides recommended amounts of such nutrients for dogs and cats. Vitamins generally useful as food additives include vitamin A, vitamin B1, vitamin B2, vitamin B6, vitamin B12, vitamin D, biotin, vitamin K, folic acid, inositol, niacin, and pantothenic acid. Minerals and trace elements useful as food additives include calcium, phosphorus, sodium, potassium, magnesium, copper, zinc, chloride, iron, selenium, iodine, and iron.

Carnitine, or L-carnitine, is a vitamin-like compound synthesized in the body from lysine and methionine. Carnitine may be naturally present in ingredients of the present invention, or carnitine may be added to the compositions.

The compositions of the present invention also may contain one or more minerals and/or trace elements. One particular trace element is manganese. Manganese is essential to a host of enzymes as a cofactor, which may regulate the metabolism of foods, including proteins, fats, and carbohydrates. Such enzymes may include oxidoreductases, transferases, hydrolases, lyases, isomerases, ligases, lectins, and integrins. Manganese also affects bone development and neurological function. Manganese may be naturally present in the components of the compositions, or it may be added to compositions. Methods of measuring manganese content in a composition are well known to those of skill in the art.

In one embodiment of the present invention, the compositions may further comprise mineral nutrients. Examples of mineral nutrients include, but are not limited to potassium chloride, calcium carbonate, and magnesium sulfate. Such mineral nutrients add important minerals to the composition, which facilitate the development and maintenance of strong, healthy bones, muscles and cartilage of animals. In particular, magnesium is important for facilitating absorption and utilization of vitamins and minerals including, but not limited to, Vitamin C, Vitamin E, calcium, phosphorus, sodium, and potassium. In one embodiment, the levels of magnesium in the compositions of the present invention can vary from about 0.02% to about 0.25% (w/w). In specific embodiments, the levels of magnesium in the compositions of the present invention are from about 0.04% to about 0.2% (w/w), from about 0.06% to about 0.15% (w/w) or from about 0.06% to about 0.1% (w/w). In one specific embodiment the levels of magnesium are at least about 0.06%.

Other mineral nutrients include, but are not limited to, sodium. The sodium can be from any source, including but not limited to, sodium chloride that is added to the compositions. Additionally, sodium may also come from other sources within the compositions such as, but not limited to, meat, poultry, fish and eggs. In one embodiment, the levels of sodium in the compositions of the present invention can vary from about 0.1% to about 0.75% (w/w). In another specific embodiment, the levels of sodium in the compositions of the present invention are from about 0.1% to about 0.5% (w/w).

Alternatively or additionally, the compositions may comprise wax. For example, illustrative waxes include paraffin wax, beeswax (e.g., white or yellow), carnuba wax, candellila wax, microcrystalline wax, rice bran wax, cetyl ester wax, and emulsifying wax. Alternatively or additionally, the compositions may comprise a polysaccharide such as shellac or chitin. Any of a variety of other lipid sources may additionally or alternatively be utilized as part or all of the lipid component herein.

In certain embodiments, the food compositions may contain additional ingredients such as fillers, palatability enhancers, binding agents, flavors, stabilizers, emulsifiers, sweeteners, colorants, buffers, salts, coatings, and the like known to skilled artisans. Stabilizers include substances that tend to increase the shelf life of the composition such as preservatives, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches. Specific amounts for each composition component, food ingredient, and other ingredients will depend on a variety of factors such as the particular components and ingredients included in the composition; the species of animal; the animal's age, body weight, general health, and diet. Therefore, the component and ingredient amounts may vary widely and may deviate from the preferred proportions described herein.

The pet food compositions may comprise a sweetener component. The sweetener component, as defined herein, is a monosaccharide, disaccharide, complex carbohydrate, or any mixture thereof. The optional sweeteners, which may be used in the foods or supplements of the present invention include, but are not limited to, saccharin, aspartame, cyclamates, acesulfame K, neohesperidin dihydrochalcone, other super sweeteners, and mixtures thereof, which may be added to the carrier in amounts sufficiently low so as not to chemically interact with the main ingredients of the nutritional supplement.

Carbohydrate may be supplied by any of a variety of sources known by those skilled in the art, including oat fiber, cellulose, peanut hulls, beet pulp, parboiled rice, corn starch, corn gluten meal, and any combination of those sources. Grains supplying carbohydrate include, but are not limited to, wheat, corn, barley, and rice. Carbohydrate content of foods may be determined by any number of methods known by those of skill in the art. Generally, carbohydrate percentage may be calculated as nitrogen free extract (“NFE”), which may be calculated as follows: NFE=100%−moisture %−protein %−fat %−ash %−crude fiber %.

In one embodiment, the pet food compositions herein comprise a complex carbohydrate. The complex carbohydrate utilized herein is an oligosaccharide, polysaccharide, and/or carbohydrate derivative, such as (for example) an oligosaccharide and/or polysaccharide. As used herein, the term “oligosaccharide” means a digestible linear molecule having from 3 to 9 monosaccharide units, wherein the units are covalently connected via glycosidic bonds. The polysaccharides may be linear chains or branched. For example, the polysaccharide has from 9 to about 20 monosaccharide units. Carbohydrate derivatives, such as a polyhydric alcohol (e.g., glycerol), may also be utilized as a complex carbohydrate herein.

Non-limiting examples of complex carbohydrates include raffinoses, stachyoses, maltotrioses, maltotetraoses, glycogens, amyloses, amylopectins, polydextroses, and maltodextrins.

In one embodiment, the complex carbohydrate is a maltodextrin. Maltodextrins are a form of complex carbohydrate molecule which is several glucose units in length. Without intending to be limited by theory, since maltodextrins are hydrolyzed into glucose in the digestive tract, they may be utilized as an extended source of glucose. Maltodextrins may be spray-dried carbohydrate ingredients made by controlled hydrolysis of corn starch.

The pet food compositions may or may not comprise a component such as dried whey or other dairy by-products. The pet food compositions may or may not comprise a fiber. In certain embodiments, the compositions may comprise a source of supplemental fiber (i.e., fiber additional to that inherently present in, for example, sources of protein, fat, or carbohydrate).

As used herein, “fiber blend” includes a combination of soluble and insoluble fiber at a level that is between 1-5% total dietary fiber on a dry matter basis. The sources of the fiber can be combinations of cellulose, hemicelluloses, resistant starches, or oligosaccharides such as galactooligosaccharides, xylooligosaccharides, or fructooligosaccharides.

Dietary fiber refers to components of a plant that are resistant to digestion by an animal's digestive enzymes. Dietary fiber components of foods may be determined by any number of methods known by those of skill in the art, such as those published by the OMA. Dietary fiber includes soluble and insoluble fibers.

Soluble fibers are resistant to digestion and absorption in the small intestine and undergo complete or partial fermentation in the large intestine, e.g., beet pulp, guar gum, chicory root, psyllium, pectin, blueberry, cranberry, squash, apples, oats, beans, citrus, barley, or peas. Insoluble fiber may be supplied by any of a variety of sources, including cellulose, whole wheat products, wheat oat, corn bran, flax seed, grapes, celery, green beans, cauliflower, potato skins, fruit skins, vegetable skins, peanut hulls, and soy fiber. Soluble and insoluble fiber content of foods may be determined by any number of methods known by those of skill in the art. Crude fiber includes indigestible components contained in cell walls and cell contents of plants such as grains, e.g., hulls of grains such as rice, corn, and beans. Crude fiber content of foods may be determined by any number of methods known by those of skill in the art.

In certain embodiments, the fiber food ingredient is obtained from a variety of sources such as vegetable fiber sources, for example, cellulose, beet pulp, peanut hulls, and soy fiber.

The source of supplemental fiber may or may not comprise a fermentable fiber. Fermentable fibers are well-known in the art. The fermentable fiber may be any fiber source which intestinal bacteria present in the animal can ferment to produce short chain fatty acids or other metabolic components. Non-limiting examples of such fermentable fibers include beet pulp (from sugar beet), gum arabic, gum talha, psyllium, rice bran, carob bean gum, citrus pulp, pectin, fructooligosaccharide, mannanoligofructose, soy fiber, arabinogalactan, galactooligosaccharide, arabinoxylan, and mixtures thereof.

In general, fermentable fibers are not digested by mammals but may be metabolized by intestinal bacterial species, such as Bifidobacterium. However, not all intestinal bacteria can metabolize fermentable fiber. In particular, bacteria such as Salmonella, E. coli and Clostridia are unable to process such fiber to any meaningful degree. This preferential digestibility, which is applicable for fermentable fiber as a class, can be used to improve the overall bacterial flora in the small intestine of the pet. Because fermentable fibers will only feed “good” bacteria such as Lactobacillus and Bifidobacterium, the amounts of harmful bacteria such as Salmonella, E. coli and Clostridia may decrease due to a reduction in food resources. Therefore, by providing a preferred food source for beneficial bacterial species, a diet supplemented with fermentable fiber can increase “good” intestinal bacteria while reducing the amount of “bad” bacteria.

Beet pulp and fructooligosaccharide, particularly short chain oligofructose, are particularly preferred fermentable fibers for use herein. As an example, fructooliogosaccharides are naturally occurring compounds which can be found in a variety of fruits or vegetables including banana, barley, garlic, honey, onion, rye, brown sugar, tomato, asparagus, artichoke, wheat, yacon, or chicory. Fructooligosaccharide may for example be provided as chicory root, as a long chain oligofructose (e.g., inulin), or as short chain oligofructose. Particularly useful herein are fructooligosaccharide comprising at least one of 1-kestose (abbreviated as GF₂), nystose (GF₃), and 1F-beta-fructofuranosylnystose (GF₄). While fructooligosaccharides can be extracted from plants such as those mentioned herein, they can also be formed artificially by adding one, two, or three fructose units to a sucrose molecule by a B-(2-1)-glycosidic linkage of the fructose unit(s) to the fructose unit of sucrose. As an example, fructooligosaccharides are commercially available under the tradename NUTRAFLORA® from Golden Technologies Company, Incorporated (which is a short chain oligofructose comprising 1-kestose, nystose, and 1F-beta-fructofuranosylnystose). As another example, a mixture of short chain fructooligosaccharide and inulin can be PREBIO™ or a mixture of commercially available RAFTILOSE® and RAFTILINE®.

The fructooligosaccharide may be a short chain oligofructose, which will be well-known to those of ordinary skill in the art. Particularly useful herein are short chain oligofructose comprising 1-kestose (abbreviated as GF₂), nystose (GF₃), and 1F-beta-fructofuranosylnystose (GF₄). In one embodiment, the short chain oligofructose comprises from about 25% to about 45% 1-kestose, from about 25% to about 45% nystose, and from about 1% to about 20% 1F-beta-fructofuranosylnystose, by weight of the short chain oligofructose, alternatively from about 30% to about 40% 1-kestose, from about 50% to about 60% nystose, and from about 5% to about 15% 1F-beta-fructofuranosylnystose, by weight of the short chain oligofructose. As an example, short chain oligofructose is commercially available under the tradename NUTRAFLORA® from Golden Technologies Company, Incorporated (which is a short chain oligofructose comprising about 35% 1-kestose, 55% nystose, and 10% 1F-beta-fructofuranosylnystose, all by weight of the short chain oligofructose).

In one embodiment herein, the compositions may comprise at least about 0.25% total fermentable fiber, by weight of the composition. By “total fermentable fiber” it is meant that the referenced level is determined by adding the relative amounts of each fermentable fiber present in the composition. For example, wherein a composition comprises 1% fructooligosaccharide and 0.5% beet pulp, by weight of the composition, and no other fermentable fiber, the composition comprises 1.5% total fermentable fiber, by weight of the composition. Alternatively, the present compositions may comprise at least about 0.5% total fermentable fiber, at least about 1% total fermentable fiber, at least about 2% total fermentable fiber, alternatively from about 1% to about 20% total fermentable fiber, alternatively from about 1% to about 10% total fermentable fiber, alternatively from about 2% to about 10% total fermentable fiber, or alternatively from about 3% to about 8% total fermentable fiber, all by weight of the pet food composition.

In one embodiment herein, the compositions may comprise a nutraceutical. Nutraceutical as used herein means a foodstuff (as a fortified food or dietary supplement) that provides health benefits.

Additionally, vitamins and minerals can be added according to the guidelines of the Association of American Feed Control Officials (AAFCO). Such AAFCO nutrient profiles include, but are not limited to, calcium carbonate, potassium chloride, sodium chloride, choline chloride, taurine, zinc oxide, ferrous sulfate, vitamin E, vitamin A (including forms thereof, such as beta-carotene and lycopenes), vitamin B₁₂, vitamin C (including forms thereof), vitamin E (including forms thereof), vitamin D (including forms thereof, such as vitamin D₃), riboflavin, niacin, calcium pantothenate, biotin, thiamine mononitrate, copper sulfate, folic acid, and pyroxidine, alkaloids, phenols, polyphenols, carotenoids, xanthones, organosulfur, curcumin, kaempherol, astaxanthin, gamma-glutamylcysteines, catechins, pterostilbene, canthaxanthin, cysteine sulfoxides, ellagic acid, quercetin, tunaxanthin, isothiocyanates, baicalin, tocopherols, myricetin, zeaxanthin, flavonoids, resveratrol, anthocyanins, bixin, isoflavonoids, vinpocetine, flavonols, lutein, Co-Q10, proanthocyanidins, lycopene, lipoic acid and the like.

The compositions herein may comprise any of a variety of components that are sensitive to process conditions ordinarily attendant with manufacture of a pet food. For example, the integrity of such sensitive components may be preserved (either fully or partially). Non-limiting examples of sensitive components include components that exhibit more than about 10% loss (by weight) during standard extrusion processes when included within a standard, commercial pet food, alternatively more than about 20% loss, alternatively more than about 50% loss. Extrusion processes are well-known in the art.

Additional material that can be present in the composition of the present invention include minerals such as but not limited to: boron, selenium, calcium chloride, chloride, ferrous fumarate, zinc acetate, chromium, ferrous gluconate, zinc sulfate, tripicolinate, cobalt, magnesium oxide, zinc gluconate, dicalcium phosphate, copper, magnesium sulfate, ferrous sulfate, iodine, magnesium carbonate, monosodium phosphate, iron, chromium picolinate, potassium chloride, magnesium, calcium citrate, potassium citrate, manganese, calcium lactate, potassium sorbate, phosphorus, calcium gluconate, sodium bisulfate, potassium, chromium chloride, sodium hexametaphosphate, sodium, chromium nicotinate, tricalcium phosphate, zinc, chromium citrate, yeast containing any of these minerals and the like.

The optional flavorants which may be used in the foods or supplements of the present invention include, but are not limited to, peppermint, peppermint-menthol, eucalyptol wintergreen, licorice, clove, cinnamon, spearmint, cherry, lemon, orange lime, menthol and various combinations thereof.

Such additives are present in amounts that do not impair the purpose and effect provided by the invention. Examples of additives include, for example, substances with a stabilizing effect, processing aids, substances that enhance palatability, coloring substances, and substances that provide nutritional benefits.

Stabilizing substances include, for example, substances that tend to increase the shelf life of the composition. Potentially suitable examples of such substances include, for example, preservatives, antioxidants, synergists and sequesterants, packaging gases, stabilizers, emulsifiers, thickeners, gelling agents, and humectants. Examples of emulsifiers and/or thickening agents include, for example, gelatin, cellulose ethers, starch, starch esters, starch ethers, and modified starches.

Additives for coloring, palatability (“pal enhancers”), and nutritional purposes include, for example, colorants (e.g., iron oxide, such as the red, yellow, or brown forms); sodium chloride, potassium citrate, potassium chloride, and other edible salts; vitamins; minerals; and flavoring. Such additives are known in the art. See, e.g., U.S. Pat. No. 3,202,514. See also, U.S. Pat. No. 4,997,671. Flavorants include, for example, dairy product flavorants (e.g., milk or cheese), meat flavorants (e.g., bacon, liver, beef, poultry, or fish), oleoresin, pinacol, and the various flavorants identified in the trade by a FEMA (Flavor Extract Manufacturers Association) number. Flavorants help provide additional palatability, and are known in the art. See, e.g., U.S. Pat. No. 4,997,672. See also, U.S. Pat. No. 5,004,624, U.S. Pat. No. 5,114,704, U.S. Pat. No. 5,532,010, and U.S. Pat. No. 6,379,727. The concentration of such additives in the composition typically may be up to about 5% by weight. In some embodiments, the concentration of such additives (particularly where such additives are primarily nutritional balancing agents, such as vitamins and minerals) is from about 0% to about 2.0% by weight. In some embodiments, the concentration of such additives (again, particularly where such additives are primarily nutritional balancing agents) is from about 0% to about 1.0% by weight.

Supplements include, for example, a feed used with another feed to improve the nutritive balance or performance of the total. Supplements include compositions that are fed undiluted as a supplement to other feeds, offered free choice with other parts of an animal's ration that are separately available, or diluted and mixed with an animal's regular feed to produce a complete feed. Supplements may be in various forms including, for example, powders, liquids, syrups, pills, encapsulated compositions, and the like.

Treats include, for example, compositions that are given to an animal to entice the animal to eat during a non-meal time. Treats for canines include, for example, dog bones. Treats may be nutritional, wherein the composition comprises one or more nutrients, and may, for example, have a composition as described above for food. Non-nutritional treats encompass any other treats that are non-toxic. The compositions can contain additional ingredients intended to maintain or improve the health of the animal, for example, supplements, medications, herbs, holistic drugs and compositions, and the like.

The composition of the invention may include one or more additional ingredients to prevent or treat one or more diseases or conditions. The component in the diet, which accomplishes this, may be an antioxidant or mixture thereof. Examples of such materials include foods such as Ginkgo Biloba, citrus pulp, grape pomace, tomato pomace, carrot and spinach, all preferably dried as well as various other materials such as beta-carotene, selenium, coenzyme Q10 (ubiquinone), lutein, tocotrienols, soy isoflavones, S-adenosylmethionine, glutathione, taurine, N-acetylcysteine, Vitamin E, Vitamin C, alpha-lipoic acid, 1-carnitine and the like. Vitamin E can be administered as a tocopherol or a mixture of tocopherols and various derivatives thereof such as esters like vitamin E acetate, succinate, palmitate, and the like. The alpha form is preferable but beta, gamma and delta forms can be included. The d form is preferable but racemic mixtures are acceptable. The forms and derivatives will function in a Vitamin E like activity after ingestion by the pet. Vitamin C can be administered in this diet as ascorbic acid and its various derivatives thereof such as calcium phosphate salts, cholesteryl salt, 2-monophosphate, and the like which will function in a vitamin C-like activity after ingesting by the pet. They can be in any form such as liquid, semisolid, solid and heat stable form. L-carnitine can be administered in the diet and various derivatives of carnitine such as the salts such as the hydrochloride, fumarate and succinates, as well as acetylated carnitine, and the like can be used.

In certain embodiments, the compositions further include an effective amount of at least one substance selected from the group consisting of glucosamine, chondroitin, chondroitin sulfate, methylsulfonylmethane (“MSM”), creatine, antioxidants, Perna canaliculata, omega-3 fatty acids, omega-6 fatty acids, tocopherols, ascorbate, calcium, chloride, cysteine, magnesium, manganese, methionine, phosphorous, potassium, iron, essential amino acids and mixtures thereof.

As contemplated herein, functional ingredients and nutrients for use in the present invention include walnut oil, sesame oil, sunflower oil, capsibiol-T, pomegranate, magnolia, lipoic acid, vitamin C, ginger, green and black tea, optionally together with an optimal fiber blend of soluble and insoluble fibers. Juices, extracts, pulp or other forms of formulations of said ingredients are included.

In certain embodiments, the dietary food composition may further include L-tryptophan. The quantities administered in the diet, all as wt % (dry matter basis) of the diet, are calculated as the active material, per se, that is measured as free material. The maximum amounts employed should not bring about toxicity.

### Preparation of the Compositions of the Invention

The edible compositions of the invention may be prepared in a canned or wet form using conventional food preparation processes known to skilled artisans. Typically, ground animal proteinaceous tissues are mixed with the other ingredients such as fish oils, cereal grains, balancing ingredients, special purpose additives (e.g., vitamin and mineral mixtures, inorganic salts, cellulose and beet pulp, bulking agents, and the like) and water in amounts sufficient for processing. These ingredients are mixed in a vessel suitable for heating while blending the components. Heating of the mixture is effected using any suitable manner, for example, direct steam injection or using a vessel fitted with a heat exchanger. Following the addition of the last ingredient, the mixture is heated to a temperature of about 50° F. to about 212° F. Temperatures outside this range are acceptable but may be commercially impractical without use of other processing aids. When heated to the appropriate temperature, the material will typically be in the form of a thick liquid. The thick liquid is filled into cans. A lid is applied, and the container is hermetically sealed. The sealed can is then placed into conventional equipment designed to sterilize the contents. Sterilization is usually accomplished by heating to temperatures of greater than about 230° F. for an appropriate time depending on the temperature used, the composition, and similar factors. The compositions of the present invention can be added to the food compositions before, during, or after preparation.

Food compositions may be prepared in a dry form using conventional processes known to skilled artisans. Typically, dry ingredients such as animal protein, plant protein, grains, and the like are ground and mixed together. Moist or liquid ingredients, including fats, oils, animal protein, water, and the like are then added to and mixed with the dry mix. The mixture is then processed into kibbles or similar dry pieces. Kibble is often formed using an extrusion process in which the mixture of dry and wet ingredients is subjected to mechanical work at a high pressure and temperature and forced through small openings and cut off into kibble by a rotating knife. The wet kibble is then dried and optionally coated with one or more topical coatings such as flavors, fats, oils, powders, and the like. Kibble also can be made from the dough using a baking process, rather than extrusion, wherein the dough is placed into a mold before dry-heat processing.

In preparing a composition for use with the methods of the present invention, any ingredient (e.g., fish oil) generally may, for example, be incorporated into the composition during the processing of the formulation, such as during and/or after mixing of other components of the composition. Distribution of these components into the composition can be accomplished by conventional means. In one embodiment, ground animal and poultry proteinaceous tissues are mixed with the other ingredients, including fish oils, cereal grains, other nutritionally balancing ingredients, special-purpose additives (e.g., vitamin and mineral mixtures, inorganic salts, cellulose and beet pulp, bulking agents, and the like); and water that is sufficient for processing is also added.

The compositions may also be designed to be easier to chew. Canine and feline foods are typically formulated based on life stage (age), size, body composition, and breed. In the methods of this invention, some embodiments of the compositions address specific nutritional differences between super senior regular or small breed dogs, large breed dogs, and cats.

Foods of any consistency or moisture content are contemplated, e.g., the compositions of the present invention may be a moist, semi-moist, or dry animal food composition. “Moist” food refers to food that has moisture content of 60 to 90% or greater. “Dry” food refers to compositions with 3 to 11% moisture content and is often manufactured in the form of small bits or kibbles. “Semi-moist” refers to compositions with 25-35% moisture content. Also contemplated herein are compositions that may comprise components of various consistency as well as components that may include more than one consistency, for example, soft, chewy meat-like particles as well as kibble having an outer cereal component and an inner cream component as described in, e.g., U.S. Pat. No. 6,517,877.

In one embodiment, the methods relate to oral administration of a composition described herein directly to a pet. The various embodiments of the composition used in this method, including forms or the composition and levels of various components contained therein, are described in detail herein. As used herein with respect to the processes of this invention, the terms “orally administering,” “oral administration” or the like means that the pet ingests or is directed to ingest one or more compositions described herein, or the owner of such pet is directed to provide one or more compositions to the pet.

The compositions may be administered in accordance with a variety of frequencies or durations. For example, the compositions are typically administered at least once weekly, or at least three times weekly, or from once daily to about four times daily, alternately from once daily to about three times daily, alternately from once daily to about two times daily, alternately ad libitum. In order to achieve the benefits herein, it is preferred that the compositions are administered for at least about one week, alternatively at least about two weeks, alternately at least about three weeks, alternately at least about four weeks, alternately at least about 6 weeks, alternately at least about eight weeks, or in an unlimited duration.

### SPECIFIC EMBODIMENTS OF THE INVENTION

The invention is further described in the following examples. The examples are merely illustrative and do not in any way limit the scope of the invention as described and claimed.

### Example 1

### Preparation of Compositions

Four separate compositions (A-D) were prepared using standard extrusion methods. Diet A (control) was comprised of about 18.5% (w/w) protein and about 8%-10% fat. The protein source in Diet A was an equal distribution of corn and poultry meal. Diet B also comprised about 18.5% (w/w) protein and about 8%-10% fat. The protein source in Diet B was an equal distribution of corn, soybean meal and poultry meal. Diet C also comprised about 18.5% (w/w) protein and about 8%-10% fat. The protein source in Diet C was an equal distribution of corn, chicken egg and poultry meal. Diet D also comprised about 18.5% (w/w) protein and about 8%-10% fat. The protein source in Diet D was an equal distribution of corn, lamb meat and poultry meal.

TABLE 1
Nutrient Components of Compositions

| Component | | | | |
|---|---|---|---|---|
| (dry matter basis) | Diet A | Diet B | Diet C | Diet D |
| Protein (%) | 18.5 | 18.5 | 18.5 | 18.5 |
| Fat (%) | 8 | 8 | 10 | 8 |
| Ca (%) | 0.65 | 0.65 | 0.65 | 1.18 |
| P (%) | 0.56 | 0.55 | 0.55 | 0.76 |
| Mg (%) | 0.1 | 0.13 | 0.1 | 0.1 |
| Na (%) | 0.15 | 0.15 | 0.15 | 0.15 |
| K (%) | 0.65 | 0.68 | 0.65 | 0.65 |
| Vitamin E (IU/g) | 0.18 | 0.18 | 0.19 | 0.18 |
| Choline (ppm) | 1200 | 1200 | 1200 | 1200 |
| Total Fatty Acids (%) | 2.34 | 2.3 | 2.63 | 2.06 |
| Omega-3 Fatty Acids | 2.25 | 2.21 | 2.51 | 1.96 |
| (%) | | | | |
| Omega-6 Fatty Acids | 2.25 | 2.21 | 2.53 | 1.96 |
| (%) | | | | |
| Crude Fiber | 2.3 | 2.53 | 2.11 | 2.32 |

### Example 2

### Effects of a Composition Comprising Soybean Meal on Female Dogs

In the Examples that follow, forty (40) healthy beagle dogs were fed Diet A for thirty (30) days. Day 30 constituted t₀. The beagle dogs were randomized into four (4) groups of five (5) female and five (5) male dogs per group. The control group of dogs remained on the control diet for six (6) days. Day 6 constituted t₁. Blood samples were taken from the control and test animals at t₀ and t₁. Age and body weight of dogs were analyzed by ANOVA and were found to be randomly distributed among groups. Age and body weight of dogs were analyzed for correlation with metabolite data by partial least square analysis. Samples were analyzed in semi-randomized analytical sequence design (samples of each dog analyzed in subsequent slots, sex and groups randomized) with pools generated from control group samples from both time points and sexes. Raw data was normalized to median of pool per sequence to account for machine variability (pool ratios). Uni- and multi-variate quality checks were made of pool ratios. Further individual normalization was performed for t₁ to t₀ data to account for inter-individual variability. Further sex-specific normalization to median of the control group was performed to focus on diet-induced metabolic changes. Basic statistics (median, mean, minimum, maximum) were calculated on these ratios. Multivariate and t-Test analysis were performed on log-transformed ratios.

Animals were fed either control Diet A or Diet B for 6 days. Serum samples were taken at day zero (prior to administration of the compositions) and at day 6. Various metabolic status markers and/or their metabolites were analyzed via HPLC to determine if differences existed in the metabolic profiles of the experimental and control animals.

In female dogs receiving Diet B, amino acids (especially Asp, Glu, Pro, Trp) were increased in serum, compared to control animals receiving Diet A. In addition, levels of urea decreased in female animals, compared to control animals. In male dogs, the opposite effects were seen. The alterations in the metabolic profile in female dogs given Diet B indicate a promotion to an anabolic state, compared to control female dogs and male dogs. Regarding the lipid profile, glycerol all triacylglycerols (TAGs) decreased in the female dogs on Diet B, but TAGs did not change in male dogs. Progesterone was lowered in female dogs and thyroxine and flavin adenine dinucleotide (FAD) were increased in female dogs fed Diet B. The opposite effects were noted in male dogs.

### Example 3

### Effects of a Composition Comprising Eggs on Female Dogs

Animals were fed either Diet A or Diet C for 6 days. Serum samples were taken at day zero (prior to administration of the compositions) and at day 6. Various metabolic status markers and/or their metabolites were analyzed via HPLC to determine if differences existed in the metabolic profiles of the experimental and control animals.

In female dogs receiving Diet C, amino acids (especially Asp, Glu, Pro, Val) were increased in serum, compared to control animals receiving Diet A. In addition, levels of urea decreased in female animals, compared to control animals. The alterations in the metabolic profile in female dogs given Diet C indicate a promotion to an anabolic state, compared to control female dogs and to male dogs. Regarding the lipid profile, glycerol, all triacylglycerols (TAGs) as well as eicosapentaenoic acid decreased in the female dogs on Diet C, but TAGs (and cholesterol) increased in male dogs. Progesterone was lowered in female dogs and thyroxine and FAD were increased in female dogs fed Diet C. The opposite effects were noted in male dogs. Docosahexaenoic acid increased in females and males as expected from addition of eggs.

### Example 4

### Effects of a Composition Comprising Lamb Meat on Female Dogs

Animals were fed either Diet A or Diet D for 6 days. Serum samples were taken at day zero (prior to administration of the compositions) and at day 6. Various metabolic status markers and/or their metabolites were analyzed via HPLC to determine if differences existed in the metabolic profiles of the experimental and control animals.

In female dogs receiving Diet D, amino acids (especially Arg, Glu, Pro) were increased in serum, compared to control animals receiving Diet A. In addition, levels of urea decreased in female animals, compared to control animals. In male dogs, the opposite effects were seen. The alterations in the metabolic profile in female dogs given Diet D indicate a promotion to an anabolic state, compared to control female dogs and to male dogs. Regarding the lipid profile, glycerol and 5 of 6 types of triacylglycerols (TAGs) assayed decreased in the female dogs on Diet D, but TAGs did not change in male dogs. Thyroxine and FAD were increased in female dogs fed Diet D. The opposite effects were noted in male dogs.

1. A method of maintaining an anabolic state in a female animal, the method comprising administering a composition to the female animal, the composition comprising between about 15% to about 25% protein (w/w) and about 5% to about 15% fat (w/w), wherein the protein source in the composition comprises at least one source selected from the group consisting of soybean meal, chicken eggs and lamb meat.

2. The method of claim 1, wherein the concentration of protein is between about 17% and about 20%.

3. The method of claim 1, wherein the anabolic state is one in which the animal exhibits at least one of the following conditions: tissue building, mineralization of bone, increase of muscle mass, maintenance of muscle mass, weight gain or weight maintenance.

4. The method of claim 1, wherein the protein source is soybean meal.

5. The method of claim 4, wherein the protein source further comprises corn and poultry meal.

6. The method of claim 5, wherein the protein source further comprises at least one source selected from the group consisting of chicken eggs and lamb meat.

7. The method of claim 1, wherein the protein source is chicken eggs.

8. The method of claim 7, wherein the protein source further comprises corn and poultry meal.

9. The method of claim 8, wherein the protein source further comprises at least one source selected from the group consisting of soybean meal and lamb meat.

10. The method of claim 1, wherein the protein source is lamb meat.

11. The method of claim 10, wherein the protein source further comprises corn and poultry meal.

12. The method of claim 11, wherein the protein source further comprises at least one source selected from the group consisting of chicken eggs and soybean meal.

13. The method of claim 1, wherein the animal is a canine.

14. The method of claim 1, wherein the animal is a feline.

15. A method of promoting an anabolic state in a female animal, the method comprising administering a composition to the female animal, the composition comprising between about 15% to about 25% protein (w/w) and about 5% to about 15% fat (w/w), wherein the protein source in the composition comprises at least one source selected from the group consisting of soybean meal, chicken eggs and lamb meat.

16. The method of claim 15, wherein the concentration of protein is between about 17% and about 20%.

17. The method of claim 15, wherein the anabolic state is one in which the animal exhibits at least one of the following conditions: tissue building, mineralization of bone, increase of muscle mass, maintenance of muscle mass, weight gain or weight maintenance.

18. The method of claim 15, wherein the protein source is soybean meal.

19. The method of claim 18, wherein the protein source further comprises corn and poultry meal.

20. The method of claim 19, wherein the protein source further comprises at least one source selected from the group consisting of chicken eggs and lamb meat.

21. The method of claim 15, wherein the protein source is chicken eggs.

22. The method of claim 21, wherein the protein source further comprises corn and poultry meal.

23. The method of claim 22, wherein the protein source further comprises at least one source selected from the group consisting of soybean meal and lamb meat.

24. The method of claim 15, wherein the protein source is lamb meat.

25. The method of claim 24, wherein the protein source further comprises corn and poultry meal.

26. The method of claim 25, wherein the protein source further comprises at least one source selected from the group consisting of chicken eggs and soybean meal.

27. The method of claim 15, wherein the animal is a canine.

28. The method of claim 15, wherein the animal is a feline.
